(19) United States
(12) Patent Application Publication
(10) Pub. No.: US 2022/0386926 A1
(43) Pub. Date: Dec. 8, 2022

(54) METHODS AND SYSTEMS FOR ANALYZING ELECTROCARDIOGRAM (ECG) SIGNALS

(71) Applicant: Pacesetter, Inc., Sylmar, CA (US)

(72) Inventors: Fady Dawoud, Studio City, CA (US); Dean P. Andersen, San Jose, CA (US); Wenwen Li, San Jose, CA (US); Yun Qiao, Sunnyvale, CA (US)

(21) Appl. No.: 17/711,735

(22) Filed: Apr. 1, 2022

Related U.S. Application Data
(60) Provisional application No. 63/197,527, filed on Jun. 7, 2021.

(51) Int. Cl. A61B 5/308; A61B 5/352; A61B 5/355; A61B 5/353; A61B 5/283

(52) U.S. Cl. CPC A61B 5/308; A61B 5/352; A61B 5/355; A61B 5/353; A61B 5/283

(57) ABSTRACT

A computer implemented system and method include one or more processors configured to receive a plurality of electrocardiogram (ECG) signals from one or more subcutaneous implantable medical devices (IMDs) and combine at least two of the plurality of ECG signals to form a first composite ECG signal.

### RELATED APPLICATIONS

The present application claims priority to U.S. Provisional Application Ser. No. 63/197,527, filed 7 Jun. 2021, the complete subject matter of which is incorporated herein by reference in their entirety.

### BACKGROUND

Embodiments herein relate generally to implantable medical devices, and more particularly to methods and systems for analyzing electrocardiogram (ECG) signals.

Implantable medical devices (IMD) include pacemakers, cardioverters, cardiac rhythm management devices, defibrillators, whether lead-based or leadless, or the like. Each product is implanted in an individual to treat heart ailments and conditions through an electronically operated device. Many implantable medical products store a large amount of biological data from sensors.

A variety of sensors, the most basic being electrical sensing of the heart, are constantly being monitored and stored in memory. Noise sources such as alternating current (AC) voltage sources can introduce noise that is close or in the band of frequencies that are of interest to signals that are being monitored. Such noise can cause incorrect signal interpretation and interfere with the general operation of the IMDs. Filters are utilized to block this extrinsic noise from interfering with signal interpretation performed by the IMD.

Subcutaneous ECG signals exhibit characteristics that can present challenges for reliable cardiac beat-by-beat sensing, such as variable amplitude of R-waves and T-waves, which can, in turn, lead to inappropriate sensing and therapy. Known vascular IMDs typically rely on a primary channel for sensing.

### SUMMARY

A need exists for a system and a method for providing reliable subcutaneous ECG signals for analysis. Further, a need exists for a system and a method for improved cardiac sensing.

With those needs in mind, a computer implemented method, under control of one or more processors, where the one or more processors are configured with specific executable instructions, the computer implemented method including receiving a plurality of electrocardiogram (ECG) signals from one or more subcutaneous implantable medical devices (IMDs), and combining at least two of the plurality of ECG signals to form a first composite ECG signal.

In at least one example, the computer implemented method further includes passing at least one of the plurality of ECG signals through a gain channel. For example, the method includes passing each of the plurality of ECG signals through a respective gain channel.

In at least one example, the computer implemented method further includes passing at least one of the plurality of ECG signals through a filter. For example, the method includes passing each of the plurality of ECG signals through a respective filter.

In at least one example, the computer implemented method further includes passing at least one of the plurality of ECG signals through a delay. For example, the method includes passing each of the plurality of ECG signals through a respective delay.

In at least one example, the combining includes adding the at least two of the plurality of ECG signals together to form the composite ECG signal. As another example, the combining includes subtracting a first ECG signal from a second ECG signal to form the composite ECG signal.

In at least one example, the receiving and the combining occur within the one or more IMDs. As another example, the receiving and the combing occur remote from the one or more IMDs.

In at least one example, the plurality of ECG signals are measured at different far-field locations with respect to a heart.

In at least one example, the combining includes combining at least two of the plurality of ECG signals to form a second composite ECG signal that differs from the first composite ECG signal. As a further example, the method includes determining which of the first composite ECG signal and the second composite ECG signal has one or more of a larger R-wave/T-wave peak ratio, a larger P-wave, or a larger T-wave.

In at least one embodiment, the combining includes cycling through pairs of one or more of vectors, gains, filters, or delays to determine one or more of a greatest R-T peak ratio, a largest P-peak amplitude, or a largest T-wave amplitude.

Certain embodiments of the present disclosure provide a system, including one or more processors, and a memory coupled to the one or more processors. The memory stores program instructions. The program instructions are executable by the one or more processors to: receive a plurality of electrocardiogram (ECG) signals from one or more subcutaneous implantable medical devices (IMDs), and combine at least two of the plurality of ECG signals to form a first composite ECG signal.

### BRIEF DESCRIPTION OF THE DRAWINGS

FIG. 1A illustrates a graphical representation of a heart with an implantable medical device (IMD) for reducing false declarations of cardiac events due to undersensing or oversensing of R-waves in accordance with embodiments herein.

FIG. 1B illustrates a graphical representation of a subcutaneous implantable medical system that is configured to implement the methods described herein and apply therapy to a heart.

FIG. 1C illustrate a graphical representation of a subcutaneous implantable medical system that is configured to implement the methods described herein and apply therapy to a heart.

FIG. 2A illustrates a block diagram of an IMD formed in accordance with embodiments herein.

FIG. 2B illustrates a block diagram of a microcontroller of an IMD formed in accordance with embodiments herein.

FIG. 2C illustrates a block diagram of a microcontroller of an IMD formed in accordance with embodiments herein.

FIG. 2D illustrates a block diagram of a microcontroller of an IMD formed in accordance with embodiments herein.

FIG. 3 illustrates a chart of a plurality of ECG signals and a composite ECG signal, according to an embodiment of the present disclosure.

FIG. 4 illustrates a flow chart of a method of providing a composite ECG signal, according to an embodiment of the present disclosure.

FIG. 5 illustrates a schematic block diagram of external devices (EDs) and networks in accordance with embodiments herein.

FIG. 6 illustrates a schematic block diagram of a distributed processing system in accordance w502ith embodiments herein.

### DETAILED DESCRIPTION

It will be readily understood that the components of the embodiments as generally described and illustrated in the Figures herein, may be arranged and designed in a wide variety of different configurations in addition to the described example embodiments. Thus, the following more detailed description of the example embodiments, as represented in the Figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, etc. In other instances, well-known structures, materials, or operations are not shown or described in detail to avoid obfuscation. The following description is intended only by way of example, and simply illustrates certain example embodiments.

The methods described herein may employ structures or aspects of various embodiments (e.g., systems and/or methods) discussed herein. In various embodiments, certain operations may be omitted or added, certain operations may be combined, certain operations may be performed simultaneously, certain operations may be performed concurrently, certain operations may be split into multiple operations, certain operations may be performed in a different order, or certain operations or series of operations may be re-performed in an iterative fashion. It should be noted that, other methods may be used, in accordance with an embodiment herein. Further, wherein indicated, the methods may be fully or partially implemented by one or more processors of one or more devices or systems. While the operations of some methods may be described as performed by the processor(s) of one device, additionally, some or all of such operations may be performed by the processor(s) of another device described herein.

Certain embodiments of the present disclosure provide systems and methods for combining ECG signals measured from a plurality of body location, such as via a plurality of subcutaneous IMDs, in order to produce a composite ECG signal that is less susceptible to inappropriate sensing (such as under-sensing or over-sensing), and better capable of detecting cardiac ventricular events (for example, R-waves).

### Terms

The terms “posture” and “patient posture” refer to postural states and/or activity levels of a patient including supine, laying on a right side, laying on a left side, sitting, standing, isometric arm exercises (e.g., pushing, pulling, and the like), ballottement, chest thump, device pressure (e.g., top, mid, and base), arm flap, handshake, and the like.

The term “activity level” refers to types of activity currently experienced by a patient, including stationary state, rest state, exercise state, walking state, and the like.

The terms “cardiac activity signal”, “cardiac activity signals”, “CA signal” and “CA signals” (collectively “CA signals”) are used interchangeably throughout to refer to an analog or digital electrical signal recorded by two or more electrodes positioned subcutaneous or cutaneous, where the electrical signals are indicative of cardiac electrical activity. The cardiac activity may be normal/healthy or abnormal/arrhythmic. Non-limiting examples of CA signals include ECG signals collected by cutaneous electrodes, and electrogram (EGM) signals collected by subcutaneous electrodes and/or by electrodes positioned within or proximate to the heart wall and/or chambers of the heart.

The terms “beat” and “cardiac event” are used interchangeably and refer to both normal and/or abnormal events.

The terms “normal” and “sinus” are used to refer to events, features, and characteristics of, or appropriate to, a heart's healthy or normal functioning.

The terms “abnormal,” or “arrhythmic” are used to refer to events, features, and characteristics of, or appropriate to, an unhealthy or abnormal functioning of the heart.

The term “real-time” refers to a time frame contemporaneous with normal or abnormal episode occurrences. For example, a real-time process or operation would occur during or immediately after (e.g., within minutes or seconds after) a cardiac event, a series of cardiac events, an arrhythmia episode, and the like.

The term “COI” refers to a characteristic of interest within CA signals. Non-limiting examples of COI from a PQRST complex, include an R-wave, P-wave, T-wave and isoelectric segments. Non-limiting examples of COI from CA signals collected at an individual electrode(s) include a sensed event (e.g., an intrinsic event or evoked response). The COI may correspond to a peak of an individual sensed event, R-wave, an average or median P, R or T-wave peak and the like.

The term “adaptive”, as used in connection with a sensitivity profile, sensitivity limit, sensitivity level or other sensing parameters, refers to an ability of the processes herein to modify the value of sensitivity and/or sensing parameters based on COI within the CA signals exceeding a COI threshold and determining that one or more of a patient posture or a respiration cycle at least in part caused the COI to exceed the COI threshold. The sensitivity profile parameters may include refractory period, start sensitivity, decay delay, sensitivity limit, slope of sensitivity decay, etc.

The term “sensitivity level”, as used herein, refers to a threshold that an input CA signal must exceed for an implantable device to identify a CA signal feature of interest (e.g., an R-wave). As one non-limiting example, software may be implemented using a programmed sensitivity level to declare an R-wave to be detected when the input CA signal exceeds the current programmed sensitivity level. In response, the software declares a device documented feature (e.g., R-wave) marker. The sensitivity level may be defined in various manners based on the nature of the CA signals. For example, when the CA signals measure electrical activity in terms of millivolts, the sensitivity level represents a millivolt threshold. For example, when a cardiac beat with a 0.14 mV amplitude is sensed by a device hardware, and R-wave may be detected when the current sensitivity level is programmed to 0.1 mV. However, when the sensitivity level is programmed to 0.15 mV or above, a cardiac beat with amplitude of 0.14 mV will not be detected as an R-wave. Embodiments herein determine an adaptive sensitivity limit and sensitivity profile for the sensitivity level.

The term “obtains” and “obtaining”, as used in connection with data, signals, information and the like, include at least one of i) accessing memory of an external device or remote server where the data, signals, information, etc. are stored, ii) receiving the data, signals, information, etc. over a wireless communications link between the IMD and a local external device, and/or iii) receiving the data, signals, information, etc. at a remote server over a network connection. The obtaining operation, when from the perspective of an IMD, may include sensing new signals in real time, and/or accessing memory to read stored data, signals, information, etc. from memory within the IMD. The obtaining operation, when from the perspective of a local external device, includes receiving the data, signals, information, etc. at a transceiver of the local external device where the data, signals, information, etc. are transmitted from an IMD and/or a remote server. The obtaining operation may be from the perspective of a remote server, such as when receiving the data, signals, information, etc. at a network interface from a local external device and/or directly from an IMD. The remote server may also obtain the data, signals, information, etc. from local memory and/or from other memory, such as within a cloud storage environment and/or from the memory of a workstation or clinician external programmer.

The abbreviations “RA”, “LA”, “RV” and “LV” refer to the right atrium, left atrium, right ventricle and the left ventricle respectively.

The term “subcutaneous” shall mean below the skin, but not intravenous. For example, a subcutaneous electrode/lead does not include an electrode/lead located in a chamber of the heart, in a vein on the heart, or in the lateral or posterior branches of the coronary sinus.

The term “marker” refers to data and/or information identified from CA signals that may be presented as graphical and/or numeric indicia indicative of one or more features within the CA signals and/or indicative of one or more episodes exhibited by the cardiac events. Markers may be superimposed upon CA signals or presented proximate to, and temporally aligned with, CA signals. Non-limiting examples of markers may include R-wave markers, noise markers, activity markers, interval markers, refractory markers, P-wave markers, T-wave markers, PVC markers, sinus rhythm markers, AF markers and other arrhythmia markers. As a further non-limiting example, basic event markers may include “AF entry” to indicate a beginning of an AF event, “in AF” to indicate that AF is ongoing, “AF exit” to indicate that AF has terminated, “T” to indicate a tachycardia beat, “B” to indicate a bradycardia beat, “A” to indicate an asystole beat, “VS” to indicate a regular sinus beat, “Tachy” to indicate a tachycardia episode, “Brady” to indicate a Bradycardia episode, “Asystole” to indicate an asystole episode, “Patient activated” to indicate a patient activated episode. An activity marker may indicate activity detected by activity sensor during the CA signal. Noise markers may indicate entry/start, ongoing, recovery and exit/stop of noise. Markers may be presented as symbols, dashed lines, numeric values, thickened portions of a waveform, and the like. Markers may represent events, intervals, refractory periods, ICM activity, and other algorithm related activity. For example, interval markers, such as the R-R interval, may include a numeric value indicating the duration of the interval. The AF markers indicate atrial fibrillation rhythmic.

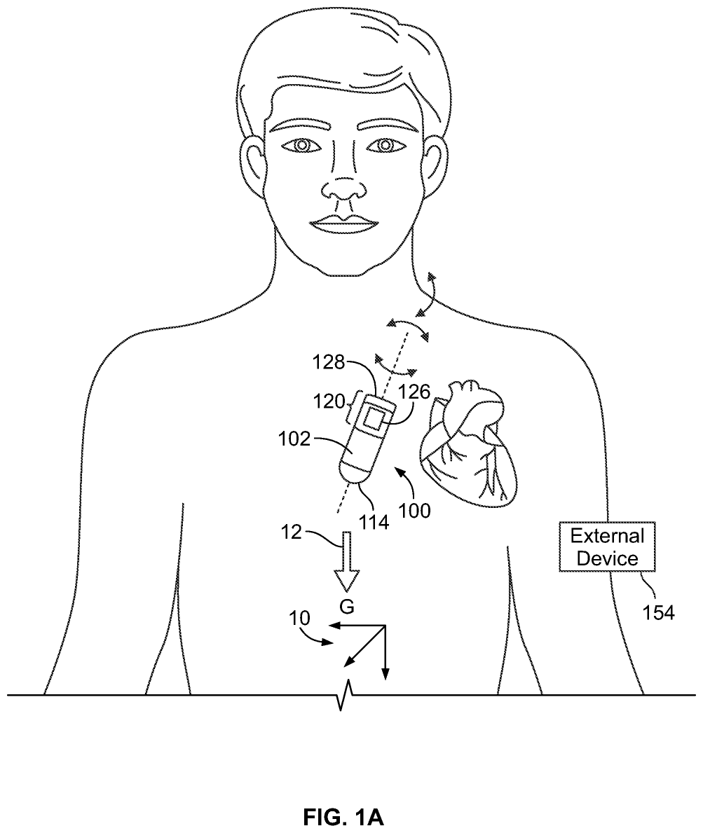

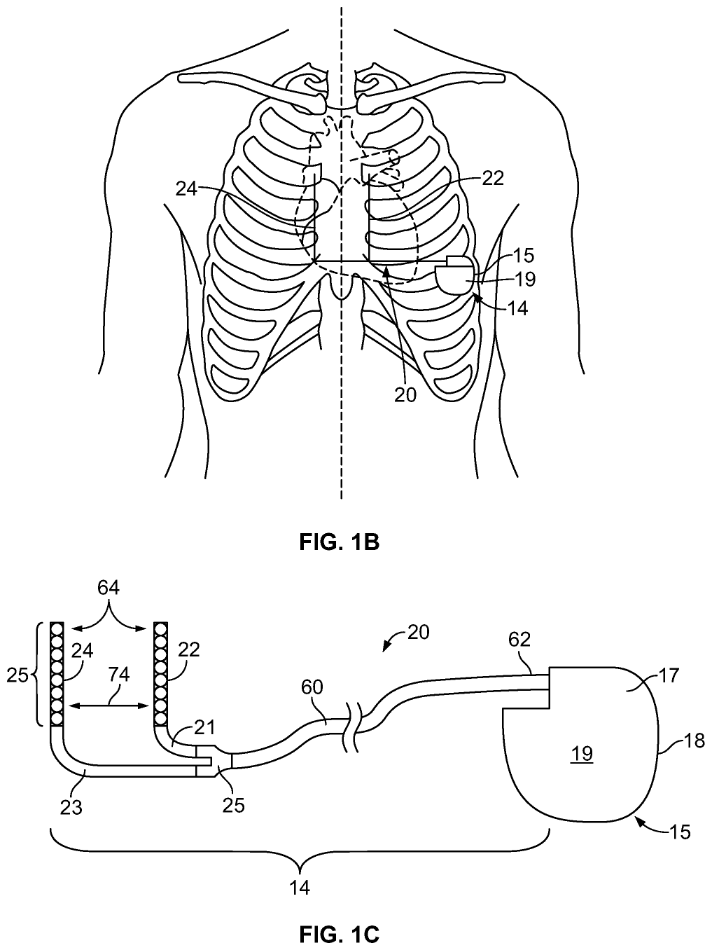

FIG. 1A illustrates an implantable medical device (IMD) 100 intended for subcutaneous implantation at a site near the heart. The IMD 100 includes a pair of spaced-apart sense electrodes 114, 126 positioned with respect to a housing 102. The sense electrodes 114, 126 provide for detection of far field electrogram signals. Numerous configurations of electrode arrangements are possible. For example, the electrode 114 may be located on a distal end of the IMD 100, while the electrode 126 is located on a proximal side of the IMD 100. Additionally, or alternatively, electrodes 126 may be located on opposite sides of the IMD 100, opposite ends or elsewhere. The distal electrode 114 may be formed as part of the housing 102, for example, by coating all but a portion of the housing with a nonconductive material such that the uncoated portion forms the electrode 114. In this case, the electrode 126 may be electrically isolated from the housing 102 electrode by placing it on a component separate from the housing 102, such as the header 120. Optionally, the header 120 may be formed as an integral portion of the housing 102. The header 120 includes an antenna 128 and the electrode 126. The antenna 128 is configured to wirelessly communicate with an external device 154 in accordance with one or more predetermined wireless protocols (e.g., Bluetooth, Bluetooth low energy, Wi-Fi, etc.).

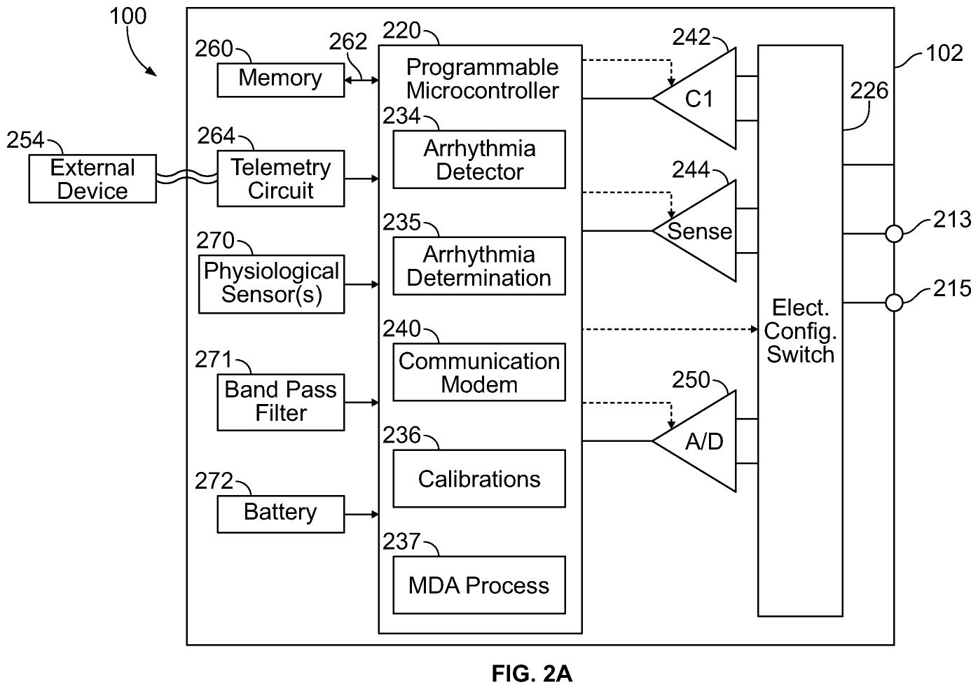

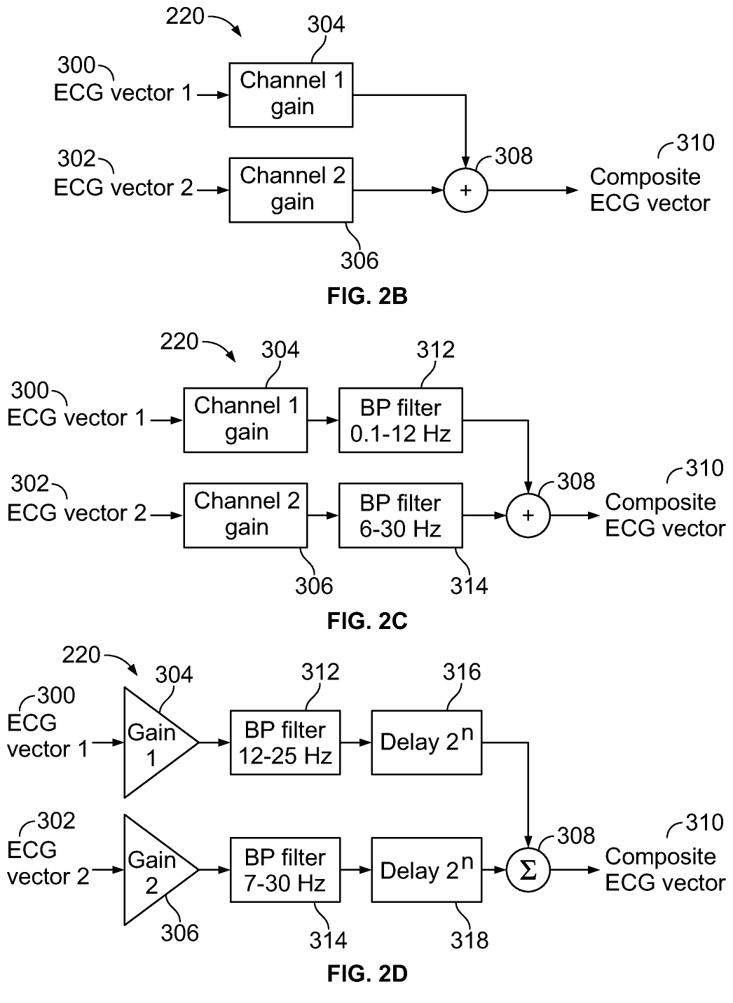

The housing 102 includes various other components such as: sense electronics for receiving signals from the electrodes, a microprocessor for analyzing the far field CA signals, including assessing the presence of R-waves in cardiac beats occurring while the IMD is in different IMD locations relative to gravitational force, a loop memory for temporary storage of CA data, a device memory for long-term storage of CA data, sensors for detecting patient activity, including an accelerometer for detecting acceleration signatures indicative of heart sound, and a battery (such as the battery 272 shown in FIG. 2A) for powering components.

In at least some embodiments, the IMD 100 is configured to be placed subcutaneously utilizing a minimally invasive approach. Subcutaneous electrodes are provided on the housing 102 to simplify the implant procedure and eliminate a need for a transvenous lead system. The sensing electrodes may be located on opposite sides of the device and designed to provide robust episode detection through consistent contact at a sensor-tissue interface. The IMD 100 may be configured to be activated by the patient or automatically activated, in connection with recording subcutaneous ECG signals.

The IMD 100 senses far field, subcutaneous CA signals, processes the CA signals to detect arrhythmias and if an arrhythmia is detected, automatically records the CA signals in memory for subsequent transmission to an external device 154.

The IMD 100 is implanted in a position and orientation such that, when the patient stands, the IMD 100 is located at a reference position and orientation with respect to a global coordinate system 10 that is defined relative to a gravitational direction 12. For example, the gravitational direction 12 is along the Z-axis while the X-axis is between the left and right arms.

As explained herein, the IMD 100 includes electrodes that collect cardiac activity (CA) signals in connection with multiple cardiac beats and in connection with different IMD locations (e.g., different positions and/or different orientations). The IMD may change location within a subcutaneous pocket relative to an initial implant position through translation and/or rotation, such as i) moving up and down (elevating/heaving) within the subcutaneous pocket; ii) moving left and right (strafing/swaying); iii) moving forward and backward (walking/surging); iv) swiveling left and right (yawing); v) tilting forward and backward (pitching); and pivoting side to side (rolling). The IMD 100 also includes one or more sensors to collect device location information indicative of movement of the IMD 100 along one or more degrees of freedom, namely translational motion along X, Y, and Z directions, and/or rotationally motion along pitch, yaw and/or roll directions.

The IMD 100 also includes one or more sensors to collect acceleration signatures that are indicative of heart sounds produced at different points in a cardiac cycle.

FIGS. 1B and 1C illustrate a graphical representation of a subcutaneous implantable medical system that is configured to implement the methods described herein and apply therapy to a heart. FIG. 1B illustrates a torso of a patient to show the rib cage and a general outline of the heart and greater vessels. In particular embodiments, the system may apply high voltage defibrillation shocks, as well as other general arrhythmia therapy, such as pacing therapy, cardiac resynchronization therapy (CRT), and the like. The system includes a subcutaneous implantable medical device (SIMD) 14 that is configured to be implanted in a subcutaneous area exterior to the heart. The SIMD 14 is an example of the IMD 100 shown in FIGS. 1A. In at least one embodiment, the system is entirely or fully subcutaneous. As shown in FIG. 1B, the SIMD 14 is positioned within a lateral region, such as along the left side of the rib cage under the left arm. The SIMD 14 may be positioned relative to a vertical direction substantially aligned with the apex of the heart. The SIMD 14 is configured to deliver various arrhythmia therapies, such as defibrillation therapy, pacing therapy, anti-tachycardia pacing therapy, cardioversion therapy, and the like. It is contemplated, however, that system may include other components. For example, alternative embodiments may include a transvenous lead or a leadless electrode in addition to the structures in FIG. 1B.

The lead 20 includes one or more electrodes 22, 24 that are used for providing electrical shock for defibrillation. Optionally, the lead 20 may include one or more sensing electrodes. The pulse generator 15 may be implanted subcutaneously and at least a portion of the lead 20 may be implanted subcutaneously. In particular embodiments, the SIMD 14 is an entirely or fully subcutaneous SIMD. The pulse generator 15 may be positioned at a lateral position or below an apex of the heart.

With reference to FIG. 1C, the lead 20 includes an elongated lead body 60 that extends from a proximal end 62 to a distal tip 64. The pulse generator 15 includes a housing 18 that is configured to be active to form a pulse-generator (PG) electrode 19. The pulse generator 15 also includes a header 17 mounted to the housing 18. The header 17 is configured to receive and be connected to the proximal end 62 of the lead body 60. The proximal end 62 may include one or more contacts (not shown) that electrically engage respective terminals (not shown) in the header 17 of the pulse generator 15.

The lead body 60 may include one or more distal branches 21, 23 that separate from a splitting connector 25, where the distal branches 21, 23 each include a corresponding one of the electrodes 22, 24, which are separated by distance 74. The splitting connector 25 may be configured in different shapes and different manners. For example, the splitting connector 25 may be formed as a Y-connector, a T-connector and the like. The splitting connector 25 may be formed as part of a monotonic unitary body structure with the lead body 60 and distal branches 21, 23.

As shown, the lead body 60 includes two distal branches 21, 23 and two electrodes 22, 24, although it is recognized that no branch, more than two branches and more than two electrodes may be provided on the lead body 60. Additionally, or alternatively, two or more separate leads 20 may be provided, with each lead 20 having a single distal segment and single electrode provided thereon. For example, the electrodes 22 and 24 may be provided on separate leads that are individually joined to the header 17. Optionally, a single lead 20 with a single electrode 22 or 24 may be used.

The electrodes 22, 24 may be referred to as first and second electrodes 22, 24 that are coupled to be electrically common with one another. The first and second electrodes 22, 24 are elongated along corresponding longitudinal axes. The first and second electrodes 22, 24 may be positioned in a dual parasternal combination extending in a common direction and spaced apart. The positioning operation may comprise positioning the first and second electrodes 22, 24 along opposite sides of the sternum, or positioning the first and second electrodes 22, 24 on a common side of the sternum. The anterior positioning operation may comprise positioning the second electrode proximate to a lower end of the sternum and orienting the second electrode to extend in a direction non-parallel to a direction of the first electrode, and locating the second electrode at a position, relative to a midline of the sternum, that is vertically below the first electrode. The non-parallel direction may orient a longitudinal axis of the second electrode perpendicular to a longitudinal axis of the first electrode.

With reference to FIG. 1B, the first electrode 22 may be positioned along a left side of the anterior region of the chest adjacent to the sternum. The second electrode 24 may be positioned along a right side of the anterior region of the chest adjacent to the sternum. Optionally, the leads may be provided in different configurations, different locations and different combinations other than shown.

FIG. 2A shows an example block diagram of the IMD 100 formed in accordance with embodiments herein. The IMD 100 may be implemented to monitor ventricular activity alone, or both ventricular and atrial activity through sensing circuit. The IMD 100 has a housing 102 to hold the electronic/computing components. The housing 102 (which is often referred to as the “can,” “case,” “encasing,” or “case electrode”) may be programmably selected to act as an electrode for certain sensing modes. Housing 102 further includes a connector (not shown) with at least one terminal 213 and optionally additional terminals 215. The terminals 213, 215 may be coupled to sensing electrodes that are provided upon or immediately adjacent the housing 102. Optionally, more than two terminals 213, 215 may be provided in order to support more than two sensing electrodes, such as for a bipolar sensing scheme that uses the housing 102 as a reference electrode. Additionally, or alternatively, the terminals 213, 215 may be connected to one or more leads having one or more electrodes provided thereon, where the electrodes are located in various locations about the heart. The type and location of each electrode may vary.

The IMD 100 includes a programmable microcontroller 220 that controls various operations of the IMD 100, including cardiac monitoring. Microcontroller 220 includes a microprocessor (or equivalent control circuitry), RAM and/or ROM memory, logic and timing circuitry, state machine circuitry, and I/O circuitry. Microcontroller 220 includes an arrhythmia detector 234 that is configured to analyze the far field cardiac activity signals to identify the existence of an arrhythmia. The microcontroller 220 also includes arrhythmia determination circuitry 235 for analyzing the CA signals to assess a presence or absence of R-waves within the cardiac beats from a first segment of the CA signals, and detect an arrythmia based on the presence or absence of one or more R-waves from the cardiac beats within a second segment of the CA signals.

The microcontroller 220 may also include a motion data analysis (MDA) process 237 configured to identify whether a COI from a first segment of the CA signals exceeds a COI limit, analyze motion data to determine whether the at least one of the posture or the respiration cycle at least in part caused the COI to exceed the COI limit, and, based on the analyze, and adjust a CA sensing parameter utilized by the IMD 100 to detect R-waves in subsequent CA signals as described in greater detail below. Consequently, arrhythmia detection accuracy is increased and false declarations of arrythmias are reduced.

The MDA process 237 is configured to implement one or more of the operations discussed herein. The MDA process 237 is configured to be a computer implemented method for reducing false declarations of arrythmias based on oversensing or undersensing of R-waves of the CA signals. The MDA process 237 obtains CA signals, at the electrodes of the IMD 100, in connection with multiple cardiac beats and, in connection with the CA signals, obtains motion data indicative of one or more of a patient posture or a respiration cycle. The method obtains motion data at one or more physiological sensors 170 (e.g., an accelerometer) and/or via a cardiac impedance (CI) sensing circuit 242 of the IMD 100 generated during the cardiac beats. The MDA process 237 identifies whether a COI from a first segment of the CA signals exceeds a COI limit and analyzes motion data to determine whether the at least one of the posture or the respiration cycle at least in part caused the COI to exceed the COI limit. Based on the analyzing operation, the MDA process 237 automatically adjusts the CA sensing parameter utilized by the IMD 100 to detect R-waves in subsequent CA signals. The CA sensing parameter defines a sensitivity profile. The MDA process 237 automatically adjusts the CA sensing parameter by changing a sensitivity of the sensitivity profile to at least reduce false arrythmia detection due to undersensing or oversensing of R-waves. Based on the adjusted CA sensing parameter, the arrythmia determination circuitry 235 detects an arrythmia based on the presence or absence of one or more of the R-waves in at least the second segment of the CA signals.

In a basic implementation, the MDA process 237 utilizes an automatic sensing adjustment based on the sensitivity profile. The sensitivity profile is defined by sensitivity profile parameter settings corresponding to the threshold start sensitivity, decay delay parameter, sensitivity limit and slope of the sensitivity decay. Optionally, the sensitivity decay may be defined in accordance with a non-linear monotonically changing shape from the threshold start sensitivity to the sensitivity limit. The start sensitivity parameter defines a start sensitivity of the sensitivity profile. For example, the start sensitivity parameter may set start sensitivity to a percentage of the preceding R-wave peak amplitude. The refractory period/interval duration parameter defines a blanking interval beginning at a sensed R-wave, during which the processors do not search for a T-wave. The decay delay parameter defines the interval at which the sensitivity profile maintains the sensitivity level at a constant level following expiration of the refractory period before the sensitivity profile begins decreasing. When the sensitivity profile includes a linear sensitivity level decline, the decay delay rate defines a slope of the linear sensitivity level decline. The sensitivity limit defines a lowest sensitivity level (e.g., maximum resolution) that linear sensitivity decline is allowed to reach. The sensitivity parameters are initially programmed to values based on baseline motion data and, over the operation of the IMD 100, are automatically adjusted based on determining that at least one of the patient posture or the respiration cycle at least in part caused the COI of the CA signals to exceed the COI limit.

In accordance with the sensitivity profile, when the CA signal crosses the sensitivity profile at starting point, the MDA process 237 treats the point as a sensed R-wave and begins a refractory interval. No new R-wave (or T-wave) will be sensed during the refractory interval. At the end of the refractory interval, the sensitivity is adjusted to a threshold start sensitivity. The threshold start sensitivity is defined as a percentage of the peak amplitude of the QRS complex of the CA signal detected during the refractory interval. The sensing circuit 244 maintains the threshold start sensitivity for a decay delay parameter, after which the MDA process begins to monotonically decrease the sensitivity (increase the resolution) of the sensing circuit 244 as denoted by a sensitivity decay within a sensitivity profile. The sensing circuit 244 continues to decrease the sensitivity until either the sensitivity decay reaches the sensitivity limit or an amplitude of the rectified CA signal exceeds the sensor sensitivity profile, such as at a point where a new sensed R wave is detected.

The sensitivity of the sensing circuit 244 is continuously adjusted by the microcontroller 220 in accordance with the sensitivity profile over the course of an individual cardiac event. Furthermore, the MDA process 237 modifies a sensitivity of the sensitivity profile of the CA sensing parameter to at least reduce false arrythmia detection due to undersensing or oversensing R-waves on at least one of i) beat by beat, or ii) for ensembles of beats. For detection for ensembles of beats, a monitoring window (e.g., 10 s-30 s) may be implemented to ensure the posture is stable over a given ensemble of beats (e.g., a moving average over the monitoring window). For example, a patient may change posture from a standing posture to a supine posture in 1 second, but changes in R-waves resulting from the change in posture from the standing posture to the supine posture may take additional time (e.g., 3-4 seconds). In additional or alternative embodiments, the MDA process 237 may monitor undersensing or oversensing of R-waves on both i) beat by beat, and ii) for ensembles of beats, and based on the posture being stable over the monitoring window, modify the sensitivity of the sensitivity profile due to undersensing or oversensing R-waves based on the ensembles of beats. Conversely, the MDA process 237 may monitor undersensing or oversensing of R-waves on both i) beat by beat, and ii) for ensembles of beats, and based on the posture being unstable (or not being stable) over the monitoring window, modify the sensitivity of the sensitivity profile due to undersensing or oversensing R-waves on a beat by beat basis. The MDA process 237 may determine, on a beat by beat basis, whether to monitor R-waves on based on i) beat by beat, or ii) for ensembles of beats.

In accordance with embodiments herein, a sensitivity of the sensitivity profile parameters may be adjusted based on the MDA process 237 determining whether at least one of the posture or the respiration cycle at least in part caused the COI to exceed the COI limit. False arrythmia detection may occur in connection with the COI exceeding the COI limit which may arise from undersensing of R-waves and/or oversensing of R-waves (e.g., sensing noise, or P-waves, or T-waves as R-waves). For example, based on the MDA process 237 determining that at least one of the patient posture or the respiration cycle at least in part caused the COI to exceed the COI limit, the MDA process 237 automatically adjusts the sensitivity of the sensitivity profile parameters. The MDA process 237 may continue to decrease the sensitivity until either a sensitivity limit is reached, or an amplitude of the CA signals exceeds the sensing profile, such that a new sensed R-wave is detected. Additionally, or alternatively, portions of the MDA process may be implemented external to the IMD 100, such as at a local external device or remote server. The local external device and/or remote server may return, to the IMD 100, adjustments to the sensitivity profile parameters based on an externally implemented portions of the MDA process 237 determining whether at least one of the posture or the respiration cycle at least in part caused the COI to exceed the COI limit.

The microcontroller 220 may also include calibration circuitry 236 that obtains calibration acceleration signatures at an accelerometer, or physiological sensor 270 that is indicative of motion data in connection with at least one of a patient posture or a respiration cycle of a patient. For example, the postures may include supine, laying on a right side, laying on a left side, or the like. In one example, the acceleration signatures are indicative of motion data generated in connection with first and second postures of a patient. After the calibration procedure, the calibration circuitry 236 utilizes the calibration acceleration signatures to determine an axis of the accelerometer associated with a current posture. The confirmation acceleration signatures are obtained along the axis of the accelerometer in connection with obtaining motion data indicative of a posture.

Although not shown, the microcontroller 220 may further include other dedicated circuitry and/or firmware/software components that assist in monitoring various conditions of the patient's heart and managing pacing therapies.

A switch 226 is optionally provided to allow selection of different electrode configurations under the control of the microcontroller 220. The electrode configuration switch 226 may include multiple switches for connecting the desired electrodes to the appropriate I/O circuits, thereby facilitating electrode programmability. The switch 226 is controlled by a control signal 228 from the microcontroller 220. Optionally, the switch 226 may be omitted and the I/O circuits directly connected to a housing electrode.

The IMD 100 is further equipped with a communication modem (modulator/demodulator) 240 to enable wireless communication. In one implementation, the communication modem 240 uses high frequency modulation, for example using RF, Bluetooth or Bluetooth Low Energy telemetry protocols. The signals are transmitted in a high frequency range and will travel through the body tissue in fluids without stimulating the heart or being felt by the patient. The communication modem 240 may be implemented in hardware as part of the microcontroller 220, or as software/firmware instructions programmed into and executed by the microcontroller 220. Alternatively, the modem 240 may reside separately from the microcontroller as a standalone component. The modem 240 facilitates data retrieval from a remote monitoring network. The modem 240 enables timely and accurate data transfer directly from the patient to an electronic device utilized by a physician.

The IMD 100 includes the CI sensing circuit 242 selectively coupled to one or more electrodes that perform sensing operations through the switch 226 to detect impedance data. For example, the CI sensing circuit 242 is coupled to various combinations of electrodes. The CI sensing circuit 242 collects impedance data by measuring voltage potentials and generating an impedance related voltage measurement stream (also referred to as an impedance data stream) associated with a corresponding CI sensing vector. The CI sensing circuit 242 is coupled to the switch 226 which connects the CI sensing circuit 242 so that voltage signals, related to impedance, at any desired electrode may be obtained. The CI sensing circuit 242, the switch 226 and the electrodes connected thereto define one or more CI sensing channels. The CI sensing channels are utilized to obtain at least one of thoracic impedance measurements or cardiogenic impedance measurements, as impedance signatures, for respiration cycles. The impedance signature for a respiration cycle is indicative of values of the components of minute ventilation in accordance with embodiments herein.

The IMD 100 includes sensing circuit 244 selectively coupled to one or more electrodes that perform sensing operations through the switch 226 to detect CA data indicative of cardiac activity. The sensing circuit 244 may include dedicated sense amplifiers, multiplexed amplifiers, or shared amplifiers. It may further employ one or more low power, precision amplifiers with programmable gain and/or automatic gain control, bandpass filtering, and threshold detection circuit to selectively sense the features of interest. In one embodiment, switch 226 may be used to determine the sensing polarity of the CA signal by selectively closing the appropriate switches.

In the example of FIG. 2A, a single sensing circuit 244 is illustrated. Optionally, the IMD 100 may include multiple sensing circuits, similar to sensing circuit 244, where each sensing circuit is coupled to two or more electrodes and controlled by the microcontroller 220 to sense electrical activity detected at the corresponding two or more electrodes. The sensing circuit 244 may operate in a unipolar sensing configuration or a bipolar sensing configuration. Optionally, the sensing circuit 244 may be removed entirely, and the microcontroller 220 perform the operations described herein based upon the CA signals from the A/D data acquisition system 250 directly coupled to the electrodes. The output of the sensing circuit 244 is connected to the microcontroller 220 which, in turn, determines when to store the CA data of the CA signals (digitized by the ND data acquisition system 250) in the memory 260. The CA signals and motion data are analyzed to determine if the COI had exceeded the COI limit and if the at least one of the posture or the respiration cycle at least part caused the COI to exceed the COI limit.

The IMD 100 further includes an analog-to-digital ND data acquisition system (DAS) 250 coupled to one or more electrodes via the switch 226 to sample CA signals across any pair of desired electrodes. The MDA process 237 may be applied to signals from the sensing circuit 244 and/or the DAS 250.

By way of example, the external device 254 may represent a bedside monitor installed in a patient's home and utilized to communicate with the IMD 100 while the patient is at home, in bed or asleep. The external device 254 may be a programmer used in the clinic to interrogate the IMD 100, retrieve data and program detection criteria and other features. The external device 254 may be a handheld device (e.g., smartphone, tablet device, laptop computer, smartwatch and the like) that may be coupled over a network (e.g., the Internet) to a remote monitoring service, medical network and the like. The external device 254 may communicate with a telemetry circuit 264 of the IMD through a communication link 266. The external device 254 facilitates access by physicians to patient data as well as permitting the physician to review real-time CA signals while collected by the IMD 100.

The microcontroller 220 is coupled to a memory 260 by a suitable data/address bus 262. The memory 260 stores the motion data, baseline motion data sets, CA signals, as well as the markers and other data content associated with detection and determination of the arrhythmia.

The IMD 100 may further include one or more physiologic sensors 270. For example, the physiologic sensor 270 may represent one or more accelerometers, such as a three-dimensional (3D) accelerometer. The sensor 270 may utilize a piezoelectric, a piezoresistive, and/or capacitive components are commonly used to convert the mechanical motion of the 3D accelerometer into an electrical signal received by the microcontroller 220. By way of example, the 3-D accelerometer may generate three electrical signals indicative of motion in three corresponding directions, namely X, Y and Z directions. The electrical signals associated with each of the three directional components may be divided into different frequency components to obtain different types of information therefrom.

The physiologic sensor 270 collects device location information with respect to gravitational force while the IMD 100 collects CA signals in connection with multiple cardiac beats. The microcontroller 220 may utilize the signals from the physiologic sensor 270 in the manner described in U.S. Pat. No. 6,937,900, titled “AC/DC Multi-Axis Accelerometer for Determining A Patient Activity and Body Position,” the complete subject matter which is expressly incorporated herein by reference. While shown as being included within the housing 102, the physiologic sensor(s) 270 may be external to the housing 102, yet still, be implanted within or carried by the patient.

The physiologic sensor 270 may be further configured to obtain motion data in the form of acceleration signatures generated during cardiac beats. The acceleration signatures from the sensor 270 are provided to the microcontroller 220 and are analyzed by the MDA process 237. The motion data is indicative of one or more of the patient posture or the respiration cycle.

The IMD 100 can also include at least one band pass filter 271. For example, the microcontroller 220 can include at least one band pass filter 271, or otherwise be in communication with the band pass filter(s) 271.

FIG. 2B illustrates a block diagram of a microcontroller 220 of an IMD 100 (shown in FIGS. 1A-2A) formed in accordance with embodiments herein. The microcontroller 220 is configured to receive a first ECG signal 300 (for example, ECG vector 1) and a second ECG signal 302 (for example, ECG vector 2), such as from the electrodes 114, 126 of the IMD 100 shown in FIG. 1A and/or an additional IMD 100 implanted subcutaneously. In at least one embodiment, the microcontroller 220 is configured to receive additional ECG signals from the electrodes 114, 126, such as a third ECG signal, and a fourth ECG signal.

The first ECG signal 300 passes through a gain channel 304 (for example, channel 1 gain), and the second ECG signal 302 passes through a gain channel 306 (for example, channel 2 gain). After passing through the gain channels 304 and 306, the first ECG signal 300 and the second ECG signal 302 are combined by a combiner 308 to provide a composite ECG signal 310 (for example, a composite ECG vector). In at least one embodiment, the combiner 308 adds the first ECG signal 300 to the second ECG signal 302 to provide the composite ECG signal 310. As another example, the combiner 308 subtracts the first ECG signal from the second ECG signal 302 to provide the composite ECG signal 310.

As shown and described, the IMD 100 includes one or more processors, such as the microcontroller 220, such as may include the combiner 308. In at least one other embodiment, the one or more processors can be remote from the IMD 100. For example, the one or more processors can be within an external device, such as the external device 154 shown in FIG. 1A or the external device 254 shown in FIG. 2A.

Optionally, the ECG signals 300 and 302 may not pass through the gain channels 304 and 306. Instead, the first ECG signal 300 and the second ECG signal 302 can be directly received and provided to the combiner 308 without passing through gain channels.

FIG. 2C illustrates a block diagram of a microcontroller 220 of an IMD 100 (shown in FIGS. 1A-2A) formed in accordance with embodiments herein. In this embodiment, after the first ECG signal 300 passes through the first gain channel 304, the ECG signal 300 then passes through a first filter 312, such as a bandpass filter (for example, a 0.1-12 Hz bandpass filter). The first filter 312 filters the ECG signal 300 before the ECG signal 300 passes to the combiner 308. Similarly, after the second ECG signal passes through the second gain channel 306, the ECG signal 302 then passes through a second filter 314, such as a bandpass filter (for example, a 6-30 Hz bandpass filter). The second filter 314 filters the ECG signal 302 before the ECG signal 302 passes to the combiner 308. The combiner 308 then combines the first ECG signal 300 and the second ECG signal 302 to provide the composite ECG signal 310, as described above.

Optionally, the ECG signals 300 and 302 may not pass through the gain channels 304 and 306. Instead, the first ECG signal 300 and the second ECG signal 302 can be provided to the first filter 312 and the second filter 314, respectively, before passing to the combiner 308 without passing through gain channels.

FIG. 2D illustrates a block diagram of a microcontroller 220 of an IMD 100 (shown in FIGS. 1A-2A) formed in accordance with embodiments herein. In this embodiment, after the first ECG signal 300 passes through the first filter 312 (for example, a 12-25 Hz bandpass filter), the first ECG signal 300 passes through a first delay 316, before passing to the combiner 308. Similarly, after the second ECG signal 302 passes through the second filter 314, the second ECG signal 302 passes through a second delay 318, before passing to the combiner 308. The combiner 308 then combines the first ECG signal 300 and the second ECG signal 302 to provide the composite ECG signal 310, as described above.

Optionally, the ECG signals 300 and 302 may not pass through the gain channels 304 and 306 and/or the filters 312 and 314. Instead, the first ECG signal 300 and the second ECG signal 302 can be provided to the first filter 312 and the second filter 314, respectively, before passing to combiner 308 without passing through gain channels. As another example, the first ECG signal and the second ECG signal 302 can be provided directly to the first delay 316 and the second delay 318, before passing to the combiner 308 without passing through gain channels or filters.

Referring to FIGS. 2B-2D, the microcontroller 220 receives a plurality of ECG signals (for example, the first ECG signal 300 and the second ECG signal) from one or more IMDs (such as one or more subcutaneous IMDs 100, as shown in FIG. 1). The microcontroller 220 combines the plurality of ECG signals to provide and output the composite ECG signal 310. The plurality of ECG signals can pass through gain channels (such as the gain channels 304 and 306), filters (such as the filters 312 and 314), and/or delays (such as the delays 316 and 318) before passing to the combiner 308.

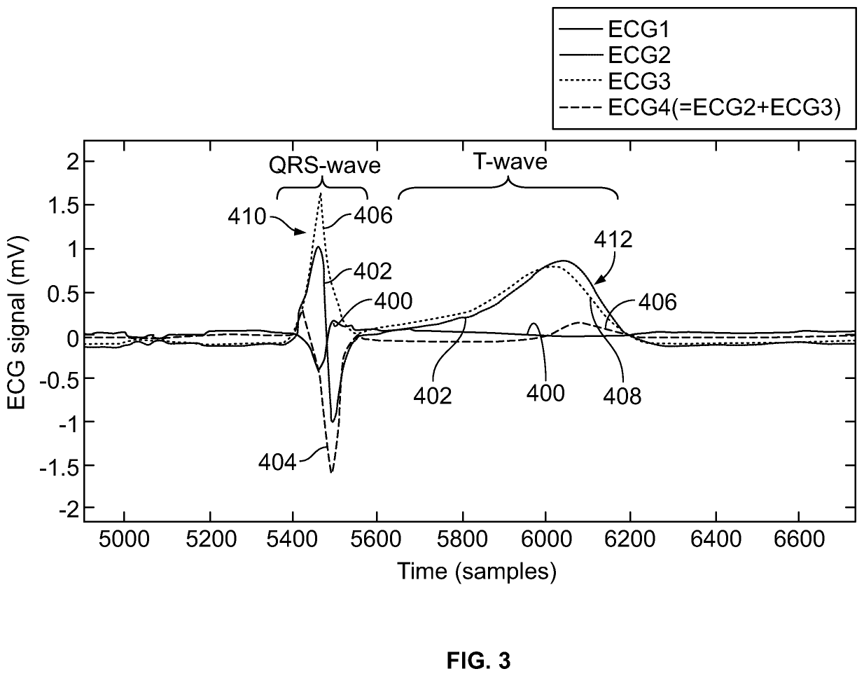

FIG. 3 illustrates a chart of a plurality of ECG signals and a composite ECG signal, according to an embodiment of the present disclosure. As shown, the ECG signals include a first ECG signal 400, a second ECG signal 402, and a third ECG signal 404. Referring to FIGS. 1A-3, the one or more processors of the IMD 100, such as the microcontroller 200, receive the first ECG signal 400, the second ECG signal 402, and the third ECG signal 404. The one or more processors of the IMD 100 combine at least two of the first ECG signal 400, the second ECG signal 402, and the third ECG signal 404 to form the composite ECG signal 406, such as a combination of the first ECG signal 400, the second ECG signal 402, and the third ECG signal 404. For example, as shown in FIG. 3, the composite ECG signal 406 is the third ECG signal 404 subtracted from the second ECG signal 402.

As another example, the composite ECG signal 406 can also include the first ECG signal 400, which can be added to or subtracted from the difference between the second ECG signal 402 and the third ECG signal 404. As another example, the composite ECG signal 406 can be a sum of two or more of the first ECG signal 400, the second ECG signal 402, and the third ECG signal 404. As another example, the composite ECG signal 406 can be a difference between the first ECG signal 400 and one of the second ECG signal 402 or the third ECG signal 404.

In at least one embodiment, the first ECG signal 400, the second ECG signal 402, and the third ECG signal 404 are all subcutaneous ECG signals, such as received from one or more IMDs 100, such as the IMD 100 shown in FIG. 1. The first ECG signal 400, the second ECG signal 402, and the third ECG signal 404 are each measured from different points away from the heart. For example, the first ECG signal 400 can be measured from the sternum, the second ECG signal 402 can be measured from a first position on a first rib, and the third ECG signal 404 can be measured from a second position, such as on the first rib or a second rib that differs from the first rib. Subcutaneous ECG signals, such as the first ECG signal 400, the second ECG signal 402, and the third ECG signal 404 (and far-field sensed cardiac signals in general) measured at a distance from cardiac tissue reflect global cardiac activation patterns and contain components of atrial and ventricular electrical depolarization (activation) and repolarization (relaxation). The ECG signals 400, 402, and 404 are measured from different locations on the human torso (for example, sternum, rib cage, and the like) with respect to the heart reflect activation pattern from those points of view and have different signal characteristics (such as QRS-wave amplitude/morphology as well as T-wave amplitude/morphology).

As shown in FIG. 3, the ECG signals 400, 402, and 404 are measured at different far-field locations with respect to the heart. While the first ECG signal 400 (green) has a smaller QRS-wave peak amplitude 410 (as compared to the QRS-wave peak amplitude 410 of the second ECG signal 402 and the third ECG signal 404), the ratio of the QRS-wave peak amplitude 410 to T-wave peak amplitude 412 for the first ECG signal 400 is the greatest, as compared to such ratio for the second ECG signal 402 and the third ECG signal 404 due to the smaller T-wave peak amplitude 412 of the first ECG signal 400. As noted, in at least one embodiment, the composite ECG signal 406 is formed (such as by the one or more processors of the IMD 100) by subtracting the third ECG signal 404 from the second ECG signal 402, or vice versa. As such, the composite ECG signal 406 exhibits an improved QRS-wave-to-T-wave amplitude peak ratio due to the T-wave amplitude peaks 412 of the composite ECG signal 406 having similar amplitudes and polarities while the QRS-waves have different amplitudes and polarities.

The composite ECG signal 406 (such as formed via one or more processors, such as the combiner 308 shown in FIGS. 2B-2D) has the advantage of capturing improved QRS-to-T wave peak ratios, as well as being robust to changes of morphology that occur, such as with ectopic arrhythmias, exercise, and change of posture. Referring again to FIGS. 2B-2D, frequency filters (such as the filters 312 and 314), gains (such as the gain channels 304 and 306), and/or delays (such as the delays 316 and 318) operate to further improve the QRS-to-T wave peak ratio of the composite ECG signal 406.

The one or more processors, such as including the combiner 308 shown in FIGS. 2B-D, can calculate ECG composite signals 406 through various combinations of the plurality of ECG signals (for example, combining at least two of the ECG signals 400, 402, and 404, such as via addition or subtraction). The one or more processors can be configured to automatically determine the resulting composite ECG signal 406 (such as from a combination of all three ECG signals 400, 402, 404, a combination of the first and second ECG signals 400 and 402, a combination of the first and third ECG signals 400 and 404, or a combination of the second and third ECG signals 402 and 404) as desired. For example, the one or more processors can determine the resulting composite ECG signal 406 having a largest R-wave/T-wave peak ratio, largest P-wave, largest T-wave, for R wave, P-wave and T-wave detection, respectively, and/or the like. As an example, there can be one composite ECG signal used for R-wave detection. After the timing of R-wave is identified, another composite ECG with a largest P-wave can be selected and used for P-wave detection. Similarly, another composite ECG can be selected and used for T-wave detection. Certain embodiments of the present disclosure allow for beat-by-beat P-wave or T-wave detection, for example.

Each of the ECG signals can be differently filtered. As such, the differently filtered ECG signals can have a different filter group delay. At lower sample rates, such as those used for storing data (for example 8 ms), the delay can be significant. Accordingly, the ECG signals can be sent to a programmable delay, such as the delays 316 and 318 shown in FIG. 2D. Moreover, the signal delay can be adjusted to further enhance QRS sensing characteristics.

Notably, there is a possibility that during ventricular fibrillation (VF) and/or ventricular tachycardia (VT), the phase of the ECG signals may match and cancel out the composite ECG signal. As such, the one or more processors can further monitor the signal amplitude and dynamically adjust composite source or revert to single vector sensing. Another embodiment may use recordings from VTNF to generate a composite signal that is suited for use during both sinus rhythm and VTNF.

In at least one embodiment, the one or more processors can identify additional composite vectors that are more favorable for detection of P-wave peaks or T-wave peaks. For example, one composite ECG signal can be used for R-wave peak detection, another composite ECG signal with the largest P-wave can be used for P-wave detection, and another composite ECG can be used for T-wave detection.

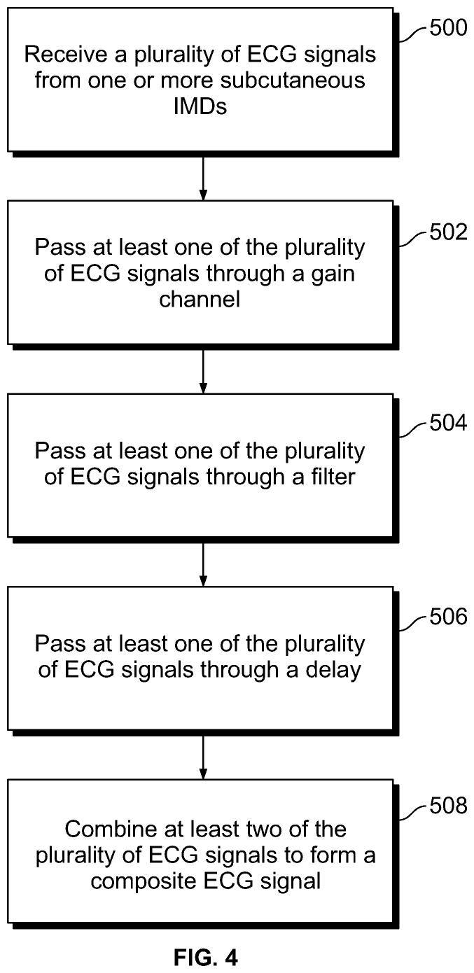

FIG. 4 illustrates a flow chart of a method of providing a composite ECG signal, according to an embodiment of the present disclosure. Referring to FIGS. 1A-4, at 500, one or more processors (such as of the IMD 100 or the external devices 154 or 254) receive a plurality of ECG signals (for example, the ECG signals 400, 402, and 404) from one or more subcutaneous IM Ds.

At 502, at least one of the plurality of ECG signals is passed through a gain channel, such as the gain channels 304 and 306. For example, each of the plurality of ECG signals is passed through a respective gain channel. Optionally, the method may not include 502.

At 504, at least one of the plurality of ECG signals is passed through a filter, such as the filters 312 and 314. For example, each of the ECG signals is passed through a respective filter. Optionally, the method may not include 504.

At 506, at least one of the plurality of ECG signals is passes through a delay, such as the delays 316 and 318. For example, each of the ECG signals is passed through a respective delay. Optionally, the method may not include 506.

At 508, at least two of the plurality of ECG signals are combined to form a composite ECG signal, such as the composite ECG signal 310. For example, the plurality of ECG signals can be added together, subtracted from one another, and/or the like to form the composite ECG signal.

In at least one embodiment, the combining 508 includes combining at least two of the plurality of ECG signals to form a second composite ECG signal that differs from the first composite ECG signal. As a further example, the method includes determining, by the one or more processors, which of the first composite ECG signal or the second composite ECG signal has one or more particular characteristics, such as one or more of a larger R-wave/T-wave peak ratio, a larger P-wave, or a larger T-wave.

In at least one embodiment, the composite ECG signal can be obtained, at least in part, by cycling through pairs of one or more of vectors, gains, filters, or delays to determine one or more of a greatest R-T peak ratio, a largest P-peak amplitude, or a largest T-wave amplitude.

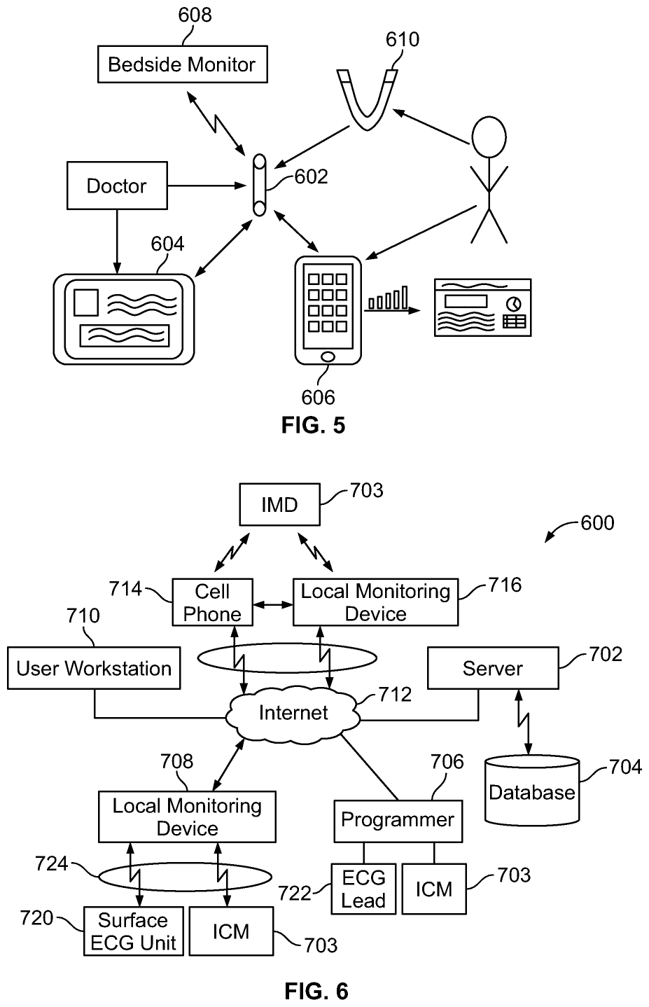

FIG. 5 illustrates a system level diagram indicating potential devices and networks that utilize the methods and systems herein. For example, an IMD 602 (for example, the IMD 100 of FIG. 1A) may be utilized to collect a cardiac activity (CA) data set, such as described herein. The IMD 602 may supply the CA data set (CA signals, sensitivity levels, and motion data) to various local external devices, such as a tablet device 604, a smart phone 606, a bedside monitoring device 608, a smart watch and the like. The devices 604-608 include a display to present the various types of the CA signals, markers, statistics, diagnostics, and other information described herein.

The IMD 602 may convey the CA data set over various types of wireless communications links to the devices 604, 606 and 608. The IMD 602 may utilize various communications protocols and be activated in various manners, such as through a Bluetooth, Bluetooth low energy, Wi-Fi, or other wireless protocol. Additionally, or alternatively, when a magnetic device 610 is held next to the patient, the magnetic field from the device 610 may activate the IMD 602 to transmit the CA data set to one or more of the devices 604-608.

FIG. 6 illustrates a distributed processing system 700 in accordance with embodiments herein. The distributed processing system 700 includes a server 702 connected to a database 704, a programmer 706, a local monitoring device 708 (for example, IMD 100) and a user workstation 710 electrically connected to a network 712. Any processor-based components (for example, workstation 710, cell phone 714, local monitoring device 716, server 702, programmer 706) may perform the processes discussed herein.

The network 712 may provide cloud-based services over the internet, a voice over IP (VoIP) gateway, a local plain old telephone service (POTS), a public switched telephone network (PSTN), a cellular phone-based network, and the like. Alternatively, the communication system may be a local area network (LAN), a medical campus area network (CAN), a metropolitan area network (MAN), or a wide area network (WAM). The communication system serves to provide a network that facilitates the transfer/receipt of data and other information between local and remote devices (relative to a patient). The server 702 is a computer system that provides services to the other computing devices on the network 712. The server 702 controls the communication of information such as CA signals, motion data, bradycardia episode information, asystole episode information, arrythmia episode information, markers, CA signal waveforms, heart rates, and device settings. The server 702 interfaces with the network 712 to transfer information between the programmer 706, local monitoring devices 708, 716, user workstation 710, cell phone 714 and database 704. The database 704 stores information such as CA data, arrythmia episode information, arrythmia statistics, diagnostics, markers, CA signal waveforms, heart rates, device settings, and the like, for a patient population. The information is downloaded into the database 704 via the server 702 or, alternatively, the information is uploaded to the server 702 from the database 704. The programmer 706 may reside in a patient's home, a hospital, or a physician's office. The programmer 706 may wirelessly communicate with the IMD 703 and utilize protocols, such as Bluetooth, GSM, infrared wireless LANs, HIPERLAN, 3G, satellite, as well as circuit and packet data protocols, and the like. Alternatively, a telemetry “wand” connection may be used to connect the programmer 706 to the IMD 703. The programmer 706 is able to acquire ECG 722 from surface electrodes on a person (e.g., ECGs), electrograms (e.g., EGM) signals from the IMD 703, and/or CA data, arrythmia episode information, arrythmia statistics, diagnostics, markers, CA signal waveforms, atrial heart rates, device settings from the IMD 703. The programmer 706 interfaces with the network 712, either via the internet, to upload the information acquired from the surface ECG unit 720, or the IMD 703 to the server 702.

The local monitoring device 708 interfaces with the communication system to upload to the server 702 one or more of the CA signals, motion data, arrythmia episode information, arrythmia statistics, diagnostics, markers, CA signal waveforms, heart rates, sensitivity profile parameter settings and detection thresholds. In one embodiment, the surface ECG unit 720 and the IMD 703 have a bi-directional connection 724 with the local RF monitoring device 708 via a wireless connection. The local monitoring device 708 is able to acquire CA signals from the surface of a person, CA data sets and other information from the IMD 703, and/or CA signal waveforms, heart rates, and device settings from the IMD 703, including after filtering of signals for environmental noise. On the other hand, the local monitoring device 708 may download the data and information discussed herein from the database 704 to the surface ECG unit 720 or the IMD 703.

The user workstation 710 may be utilized by a physician or medical personnel to interface with the network 712 to download CA signals, motion data, and other information discussed herein from the database 704, from the local monitoring devices 708, 716, from the IMD 703 or otherwise. Once downloaded, the user workstation 710 may process the CA signals and motion data in accordance with one or more of the operations described above. The user workstation 710 may upload/push settings (e.g., sensitivity profile parameter settings), IMD instructions, other information, and notifications to the cell phone 714, local monitoring devices 708, 716, programmer 706, server 702 and/or IMD 703.

As described herein, embodiments of the present disclosure provide systems and methods for providing reliable subcutaneous ECG signals for analysis. Further, a need exists for systems and methods for improved cardiac sensing.

### Closing

It should be clearly understood that the various arrangements and processes broadly described and illustrated with respect to the Figures, and/or one or more individual components or elements of such arrangements and/or one or more process operations associated of such processes, can be employed independently from or together with one or more other components, elements and/or process operations described and illustrated herein. Accordingly, while various arrangements and processes are broadly contemplated, described and illustrated herein, it should be understood that they are provided merely in illustrative and non-restrictive fashion, and furthermore can be regarded as but mere examples of possible working environments in which one or more arrangements or processes may function or operate.

As will be appreciated by one skilled in the art, various aspects may be embodied as a system, method or computer (device) program product. Accordingly, aspects may take the form of an entirely hardware embodiment or an embodiment including hardware and software that may all generally be referred to herein as a “circuit,” “module” or “system.” Furthermore, aspects may take the form of a computer (device) program product embodied in one or more computer (device) readable storage medium(s) having computer (device) readable program code embodied thereon.

Any combination of one or more non-signal computer (device) readable medium(s) may be utilized. The non-signal medium may be a storage medium. A storage medium may be, for example, an electronic, magnetic, optical, electromagnetic, infrared, or semiconductor system, apparatus, or device, or any suitable combination of the foregoing. More specific examples of a storage medium would include the following: a portable computer diskette, a hard disk, a random access memory (RAM), a dynamic random access memory (DRAM), a read-only memory (ROM), an erasable programmable read-only memory (EPROM or Flash memory), a portable compact disc read-only memory (CD-ROM), an optical storage device, a magnetic storage device, or any suitable combination of the foregoing.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of network, including a local area network (LAN) or a wide area network (WAN), or the connection may be made through other devices (for example, through the Internet using an Internet Service Provider) or through a hard wire connection, such as over a USB connection. For example, a server having a first processor, a network interface, and a storage device for storing code may store the program code for carrying out the operations and provide this code through its network interface via a network to a second device having a second processor for execution of the code on the second device.

Aspects are described herein with reference to the figures, which illustrate example methods, devices and program products according to various example embodiments. The program instructions may be provided to a processor of a general-purpose computer, special purpose computer, or other programmable data processing device or information handling device to produce a machine, such that the instructions, which execute via a processor of the device implement the functions/acts specified. The program instructions may also be stored in a device readable medium that can direct a device to function in a particular manner, such that the instructions stored in the device readable medium produce an article of manufacture including instructions which implement the function/act specified. The program instructions may also be loaded onto a device to cause a series of operational steps to be performed on the device to produce a device implemented process such that the instructions which execute on the device provide processes for implementing the functions/acts specified.

The units/modules/applications herein may include any processor-based or microprocessor-based system including systems using microcontrollers, reduced instruction set computers (RISC), application specific integrated circuits (ASICs), field-programmable gate arrays (FPGAs), logic circuits, and any other circuit or processor capable of executing the functions described herein. Additionally, or alternatively, the modules/controllers herein may represent circuit modules that may be implemented as hardware with associated instructions (for example, software stored on a tangible and non-transitory computer readable storage medium, such as a computer hard drive, ROM, RAM, or the like) that perform the operations described herein. The above examples are exemplary only, and are thus not intended to limit in any way the definition and/or meaning of the term “controller.” The units/modules/applications herein may execute a set of instructions that are stored in one or more storage elements, in order to process data. The storage elements may also store data or other information as desired or needed. The storage element may be in the form of an information source or a physical memory element within the modules/controllers herein. The set of instructions may include various commands that instruct the modules/applications herein to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It is to be understood that the above description is intended to be illustrative, and not restrictive. For example, the above-described embodiments (and/or aspects thereof) may be used in combination with each other. In addition, many modifications may be made to adapt a particular situation or material to the teachings herein without departing from its scope. While the dimensions, types of materials and coatings described herein are intended to define various parameters, they are by no means limiting and are illustrative in nature. Many other embodiments will be apparent to those of skill in the art upon reviewing the above description. The scope of the embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects or order of execution on their acts.

What is claimed is:

1. A computer implemented method, under control of one or more processors, where the one or more processors are configured with specific executable instructions, the computer implemented method comprising:
receiving a plurality of electrocardiogram (ECG) signals from one or more subcutaneous implantable medical devices (IMDs); and
combining at least two of the plurality of ECG signals to form a first composite ECG signal.

2. The computer implemented method of claim 1, further comprising passing at least one of the plurality of ECG signals through at least one of a gain channel, a filter or a delay.

3. The computer implemented method of claim 1, further comprising passing each of the plurality of ECG signals through at least one of a respective gain channel, a respective filter, or a respective delay.

4. The computer implemented method of claim 1, wherein the combining comprises adding the at least two of the plurality of ECG signals together to form the composite ECG signal.

5. The computer implemented method of claim 1, wherein the combining comprises subtracting a first ECG signal from a second ECG signal to form the composite ECG signal.

6. The computer implemented method of claim 1, wherein the receiving and the combining occur within the one or more IMDs.

7. The computer implemented method of claim 1, wherein the receiving and the combing occur remote from the one or more IMDs.

8. The computer implemented method of claim 1, wherein the plurality of ECG signals are measured at different far-field locations with respect to a heart.

9. The computer implemented method of claim 1, wherein the combining comprises combining at least two of the plurality of ECG signals to form a second composite ECG signal that differs from the first composite ECG signal.

10. The computer implemented method of claim 9, further comprising determining which of the first composite ECG signal and the second composite ECG signal has one or more of a larger R-wave/T-wave peak ratio, a larger P-wave, or a larger T-wave.

11. The computer implemented method of claim 1, wherein the combining comprises cycling through pairs of one or more of vectors, gains, filters, or delays to determine one or more of a greatest R-T peak ratio, a largest P-peak amplitude, or a largest T-wave amplitude.

12. A system, comprising:
one or more processors;
and a memory coupled to the one or more processors, wherein the memory stores program instructions, wherein the program instructions are executable by the one or more processors to:
receive a plurality of electrocardiogram (ECG) signals from one or more subcutaneous implantable medical devices (IMDs); and
combine at least two of the plurality of ECG signals to form a first composite ECG signal.

13. The system of claim 12, wherein the one or more processors are further configured to pass at least one of the plurality of ECG signals through at least one of a gain channel, filter or delay.

14. The system of claim 12, wherein the one or more processors are further configured to pass each of the plurality of ECG signals through at least one of a respective gain channel, respective filter or respective delay.

15. The system of claim 12, wherein the one or more processors are further configured to combine the at least two of the plurality of ECG signals by adding the at least two of the plurality of ECG signals together to form the composite ECG signal.

16. The system of claim 12, wherein the one or more processors are further configured to combine the at least two of the plurality of ECG signals by subtracting a first ECG signal from a second ECG signal to form the composite ECG signal.

17. The system of claim 12, wherein the one or more IMDs comprise the one or more processors.

18. The system of claim 12, wherein the one or more processors are remote from the one or more IMDs.

19. The system of claim 12, wherein the plurality of ECG signals are measured at different far-field locations with respect to a heart.

20. The system of claim 12, wherein the one or more processors are further configured to combine at least two of the plurality of ECG signals to form a second composite ECG signal that differs from the first composite ECG signal.

21. The system of claim 20, wherein the one or more processors are further configured to determine which of the first composite ECG signal and the second composite ECG signal has one or more of a larger R-wave/T-wave peak ratio, a larger P-wave, or a larger T-wave.

22. A computer implemented method, under control of one or more processors, where the one or more processors are configured with specific executable instructions, the computer implemented method comprising:
receiving a plurality of electrocardiogram (ECG) signals from one or more subcutaneous implantable medical devices (IMDs), wherein the plurality of ECG signals are measured at different far-field locations with respect to a heart;
passing each of the plurality of ECG signals through a respective gain channel;
passing each of the plurality of ECG signals through a respective filter;
passing each of the plurality of ECG signals through a respective delay; and
combining at least two of the plurality of ECG signals to form a first composite ECG signal.

23. The computer implemented method of claim 22, wherein the combining comprises combining at least two of the plurality of ECG signals to form a second composite ECG signal that differs from the first composite ECG signal.

24. The computer implemented method of claim 23, further comprising determining which of the first composite ECG signal and the second composite ECG signal has one or more of a larger R-wave/T-wave peak ratio, a larger P-wave, or a larger T-wave.
